(19) United States
(12) Patent Application Publication
(10) Pub. No.: US 2016/0270428 A1
(43) Pub. Date: Sep. 22, 2016

(54) METHOD FOR DETOXIFYING ZEARALENONE

(71) Applicant: National Taiwan University, Taipei City (TW)

(72) Inventors: Je-Ruei Liu, Taipei City (TW); Kuan-Chen Cheng, Taipei City (TW); An Lee, Taipei City (TW)

(21) Appl. No.: 14/850,565

(22) Filed: Sep. 10, 2015

(30) Foreign Application Priority Data
Mar. 17, 2015 (TW) 104108505

(51) Int. Cl. A23L 1/015; C12N 1/20; C12R 1/07

(52) U.S. Cl. CPC A23L 1/0158; C12N 1/20; C12R 1/07; A23V 2002/00

(57) ABSTRACT

The present invention provides a novel Bacillus amyloliquefaciens and a method for detoxifying zearalenone. The Bacillus amyloliquefaciens of the present invention shows high zearalenone-degrading activity, non-hemolytic and non-enterotoxin producing properties, and acidic and bile salt resistant. Moreover, B. amyloliquefaciens has the abilities of xylanase, carboxyl-methyl cellulase, amylase, and protease.

### CROSS-REFERENCE TO RELATED APPLICATION

This application claims the priority of Taiwanese patent application No. 104108505, filed on Mar. 17, 2015, which is incorporated herewith by reference.

### BACKGROUND OF THE INVENTION

1. Field of the Invention

The present invention relates to a biological method for removing mycoxtoxin, especially relates to a Bacillus amyloliquefaciens strain and use thereof for removing mycoxtoxin.

2. The Prior Arts

The feeding stuffs or food ingredients can be contaminated with zearalenone during the harvest, processing, transportation or storage period, then producing a toxic secondary metabolite which is mycoxtoxin. Mycoxtoxins are capable of causing diseases in humans and other animals, also causing the economic loss which includes the quality of cereal, safe consideration of feeding stuffs and foodstuffs and impacting on cereal trade markets. Thus, mycoxtoxins are not only bringing public health issues, but also producing economic and food problems. Mycotoxins majorly produced by mold of Aspergillus, A., Claviceps, C., Fusarium, F., and Penicillium, P., the produced mycotoxins majorly include aflatoxins, citrinin, ochratoxin, patulin, claviformin, clauacin, clavatin, expansine, mycotoxin C, ergot alkaloid, fumonisins and trichothecenes, wherein trichothecenes include T-2 toxin, deoxynivalenol, vomitoxin and zearalenone (F-2 toxin).

McNutt et al. found a sow showed false estrus as pussy, breast swelling and vaginal and rectal prolapse after feeding moldy feed in 1929. McErlean reported that swine showed the same symptoms after ate Fusarium contaminated feed barley in Ireland, 1952. But McNutt and McErlean failed to isolate the toxin. After that, the same disease had reported in France, Italy, Yugoslavia, Romania, Hungary, Denmark, Canada, etc. Andrew and Stob published that Fusarium roseum which grows in barley is capable to produce a substance having estrogen effect, i.e. zearalenone, in 1961. They also provided the isolation method and the chemical structure of zearalenone, and indicated zearalenone have anabolic and estrogen effect thereby it could serve as a substance for promoting the growth of cattle, pigs and sheep.

The feeding stuffs or food ingredients which are easily contaminated by zearalenone include corn, wheat, rice, barley, millet and oats, corn and wheat out of them are the most often contaminated by zearalenone. Zearalenone is mainly produced by Fusarium graminearum, Fusarium oxysporium, Fusarium moniliforme, Fusarium culmorum, Fusarium sporotrichiodes and Fusarium equiseti, wherein Fusarium graminearum is the main specie of zearalenone producing fungi. Zearalenone and estrogen have similar chemical structure, resulting in the reproductive failure or death in animals, also affecting the reproductive efficiency of swine. A research reported that the feeding stuffs containing 1 ppm zearalenone cause the reduction of Crude protein digestibility and feeding efficiency in swine; the feeding stuffs containing 1.1 ppm zearalenone could damage the reproduction, liver, kidney, spleen and other organs in swine. If a human intake zearalenone for the long term, abnormal spindle fibers during cell division will be made and resulting in infertility and abnormal ploidy embryos. Besides, zearalenone also causes high estrogen level, as well as dizziness, nausea, vomiting, reproductive disorders and other symptoms. Children intake zearalenone may lead to early sexual maturation, breast growth in boys and early onset mastitis. Furthermore, zearalenone also promotes the growth of human breast cancer cell. Many research prove that crops have very high zearalenone detection rate, the zearalenone detection rates of corn in North America, Japan and China are 29%, 40% and 49%, respectively; the zearalenone detection rates of wheat in Australia is 44%.

In the method of removing the mycoxtoxin in the feeding stuffs or food ingredients, biological methods are more development since the advantages of high efficiency, specificity and destroy less nutrient content. The biological methods apply to removing the mycoxtoxin content in the contaminated feeding stuffs or food ingredients are two of them. The first one is using the biological decomposition of microorganism, such as Gluconobacter oxydans could decomposition patulin, Rhodococcus erythropolis could decomposition aflatoxin B₁ and Bacillus licheniformis could decomposition ochratoxin A, zearalenone and aflatoxin B₁. The second one is using the cell walls of microorganism to adsorb the mycoxtoxin, for instant, Lactobacillus rhamnosus could adsorb aflatoxin B₁.

Bacillus amyloliquefaciens usually exist in soil or moldy crops; it is a species of bacterium in the genus Bacillus, family Bacillaceae. Bacillus amyloliquefaciens are facultative anaerobic bacterium and gram positive rods with peritrichous flagella allowing motility, producing endospore while under environmental stress. The optimal temperature for cellular growth is between 30° C. and 40° C., and can't growth in the temperature below 15° C. and higher than 50° C. The type strain of Bacillu amyloliquefaciens is Bacillu amyloliquefaciens ATCC 23350 (Fukomoto strain F). It is known that the strain is broadly used to produce amylase and protease in industry. However, there is no science paper reporting the Bacillus amyloliquefaciens has mycoxtoxin deposition ability in PubMedline database or Science Citation Index Expanded (SCIE) database of National Center for Biotechnology Information (NCBI) website.

### SUMMARY OF THE INVENTION

To solve the problem described above, the present invention provides a novel strain of Bacillus amyloliquefaciens LN for removing zearalenone, deposited in Deutsche Sammlung von Mikroorganismen and Zellkulturen GmbH and having deposit number DSM32119.

The present invention also provides a method for detoxifying zearalenone by Bacillus amyloliquefaciens LN, comprising contacting a substance containing zearalenone with the Bacillus amyloliquefaciens LN, wherein the Bacillus amyloliquefaciens LN has been deposited under DSMZ Accession number DSM32119.

In one embodiment of the present invention, the Bacillus amyloliquefaciens LN contacts with the substance containing zearalenone for a period of detoxification time, wherein the period of detoxification time is 24-36 hours. In one preferred embodiment of the present invention, the period of detoxification time is 24 hours and the zearalenone-degradation rate is 100%.

In one embodiment of the present invention, the substance containing zearalenone is corn, wheat, rice, barley, millet or oats.

In one embodiment of the present invention, the Bacillus amyloliquefaciens LN is supplemented into a food or an ingredient, and the Bacillus amyloliquefaciens LN is supplemented into with 1% concentration. In another preferred embodiment of the present invention, the Bacillus amyloliquefaciens LN is supplemented into a feeding stuff, and the Bacillus amyloliquefaciens LN is supplemented into with 1% concentration.

The present invention also provides a composition for removing zearalenone, comprising Bacillus amyloliquefaciens LN and an excipient, wherein the Bacillus amyloliquefaciens LN has been deposited under DSMZ Accession number DSM32119.

In addition to mycoxtoxin deposition ability, the Bacillus amyloliquefaciens LN of the present invention also has the abilities of xylanase, carboxyl-methyl cellulase, amylase, and protease. Moreover, the novel stain demonstrates non-hemolytic, non-enterotoxin producing properties, thus it's suitable for adding into the food or feeding stuffs, thereby eliminating the hazard of zearalenone.

### BRIEF DESCRIPTION OF THE DRAWINGS

The present invention will be apparent to those skilled in the art by reading the following detailed description of a preferred embodiment thereof, with reference to the attached drawings, in which:

FIG. 1 shows the colony morphology of Bacillus amyloliquefaciens LN;

FIG. 2 shows the Neighboring-joining phylogenetic tree based on the V1-V2 region of the 16S rRNA gene of Bacillus species. At major nodes, bootstrap percentages for 1,000 resamplings are shown. The scale bar represents 0.1 nucleotide substitutions per nucleotide position. Bacillus species include B. sonorensis, B. licheniformis, B. mojavensis, B. subtilis, B. vallismortis, B. safensis, B. stratosphericus, and B. aerophilus;

FIG. 3 shows the growth of Bacillus amyloliquefaciens LN and ATCC 23350 in LB broth containing 5 ppm of zearalenone;

FIG. 4 shows the zearalenone removal abilities of Bacillus amyloliquefaciens LN and ATCC 23350 in LB broth;

FIG. 5 shows the zearalenone removal abilities of Bacillus amyloliquefaciens LN and ATCC 23350 in phosphate buffer;

FIG. 6 shows the growth of Bacillus amyloliquefaciens LN and ATCC 23350 in corn meal medium containing 5 ppm of zearalenone;

FIG. 7 shows the zearalenone removal abilities of Bacillus amyloliquefaciens LN and ATCC 23350 in corn meal medium;

FIG. 8 shows the acid resistance of Bacillus amyloliquefaciens LN and ATCC 23350 strains;

FIG. 9 shows the bile salt resistance of Bacillus amyloliquefaciens LN and ATCC 23350 strains;

FIG. 10 shows the radial diffusion assays of amylase, xylanase, CMCase, and protease activities of Bacillus amyloliquefaciens LN and ATCC 23350 strains; and

FIG. 11 shows the extracellular xylanase, CMCase, α-amylase and protease specific activities of Bacillus amyloliquefaciens LN and ATCC 23350 strains.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

### DEFINITION

The term “Bacillus amyloliquefaciens LN” in the specification is a novel strain deposited in Deutsche Sammlung von Mikroorganismen and Zellkulturen GmbH (DSMZ) and having deposit number DSM32119. The term can be shortened to “LN strain”.

The Bacillus amyloliquefaciens LN strain is isolated from a moldy corn sample which is obtained from National Taiwan University Experimental Farm according to Petchkongkaew et al. (2008). The sample diluted with normal saline (0.85%) and then seeded in Luria-Bertani (LB) agar plate (Difco Laboratories, USA). After incubated at 37° C. for 24 hours, picked single colony and seeded in LB broth containing 5 ppm zearalenone, then shaking cultured with 250 rpm at 37° C. for 24 hours and measured zearalenone residual amount in LB broth. Among all tested colony, Bacillus amyloliquefaciens LN strain of the present invention has the best zearalenone removal ability. Further, the strain, Bacillus amyloliquefaciens ATCC 23350, was used as a control strain and purchases from Bioresource Collection and Research Center of Food Industry Research and Development Institute (Hsinchu, Taiwan).

In order to observe the colony morphology of Bacillus amyloliquefaciens LN strain, the strain seeded in LB agar plate (Merck, Germany) at 37° C. for 24 hours and then observed. In order to observe the cell morphology of Bacillus amyloliquefaciens LN strain, the strain seeded in LB broth at 37° C. for 24 hours, collected with 5000 g centrifugation, stained with Gram Staining kit (Sigma-Aldrich Co., USA) and then observed under a microscope. Then another group of the same strain was prepared, followed the method disclosed in Waldeck et al. (2006) to stain with 4′,6-diamidino-2-phenylindole (DAPI), and observed under a fluorescence microscope.

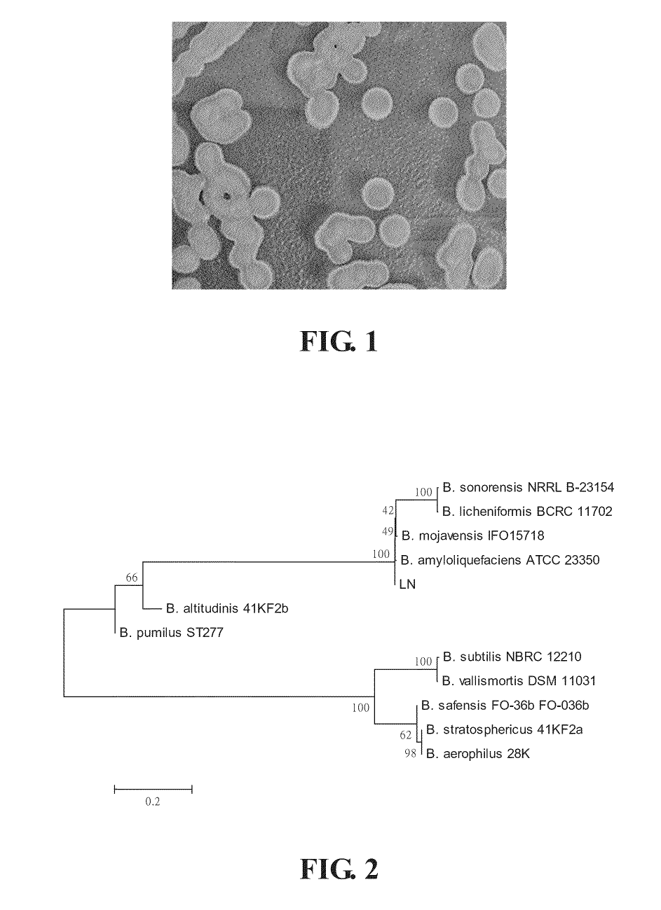

The colony morphology of Bacillus amyloliquefaciens LN strain is shown in FIG. 1. The colony surface is smooth and sticky and has protrusion shape. The cell stained with DAPI stain and observed under a phase contrast microscope and a fluorescence microscope. The cell is rod shape with peritrichous flagella allowing motility, appearing single cells or connecting several cells to form a chain. The LN strain cells stained by gram staining are purple under a microscope, indicating the LN strain is gram positive. The colony and cell morphology characteristics described above are consistency with the Bacillus amyloliquefaciens characteristics disclosed in Bergey's Manual of Systematic Bacteriology (Vos et al., 2009).

The biochemical analysis of Bacillus amyloliquefaciens LN strain is using API 50 CHB kit (bioMerieux Inc., USA), the result showed in Table 1. The difference between LN strain and Bacillus amyloliquefaciens ATCC 23350 is mainly in the usage of D-xylose, D-galactose, D-glucose, D-mannose, dulcitol, D-lactose, melibiose and N-acetylglucosamine. The two strains have 99.8% similarity compared by API software.

TABLE 1
The biochemical characteristics of Bacillus amyloliquefaciens
LN and Bacillus amyloliquefaciens ATCC 23350.

| | strain | | | strain | |
|---|---|---|---|---|---|
| substrate | LN | ATCC23350 | substrate | LN | ATCC23350 |
| Glycerol | + | + | Esculin | + | + |
| | | | ferric citrate | | |
| Erythritol | − | − | Salicin | + | + |
| D-Arabinose | − | − | D-Cellobiose | + | + |
| L-Arabinose | + | + | D-Maltose | + | + |
| D-Ribose | + | + | D-Lactose | + | − |
| D-Xylose | + | − | Melibiose | − | + |
| L-Xylose | − | − | D-Sucrose | + | + |
| D-Adonitol | − | − | D-Trehalose | + | + |
| Methyl- | − | − | Inulin | + | + |
| xylopyranoside | | | | | |
| D-Galactose | − | + | D-Melezitose | − | − |
| D-Glucose | + | − | D-Raffinose | + | + |
| D-Fructose | + | + | Amidon/Starch | + | + |
| D-Mannose | + | − | Glycogen | + | + |
| D-Sorbose | + | + | Xylitol | − | − |
| Rhamnose | − | − | Gentiobiose | − | − |
| Dulcitol | − | + | D-Turanose | − | − |
| Inositol | − | − | D-Lyxose | − | − |
| D-Mannitol | + | + | D-Tagatose | + | + |
| D-Sorbitol | + | + | D-Frucose | − | − |
| α-Methyl-D- | − | − | L-Frucose | − | − |
| mannoside | | | | | |
| α-Methyl-D- | + | + | D-Arabitol | − | − |
| glucoside | | | | | |
| N-Acetylglu- | + | − | L-Arabitol | − | − |
| cosamine | | | | | |
| Amygdalin | + | + | Gluconate | − | − |
| Arbutin | + | + | 2- | − | − |
| | | | Ketogluconate | | |
| | | | 5- | − | − |
| | | | Ketogluconate | | |

The method described in Weisburg et al. (1990) was used for molecular identification of LN strain. The DNA extract kit (Qiagen Inc., USA) was used to extract genomic DNA of LN strain. The primer pair 16S-27f (SEQ ID NO: 1) and 165-1492r (SEQ ID NO: 2) was used for polymerase chain reaction (PCR) to amplify the 16S rRNA gene sequence of LN strain. After sequencing the PCR products, choose the positions 54-510 of 16S rDNA of LN strain (V1-V3 highly variable region included) for comparing with other bacteria species. The BioEdit Sequence Alignment Editor program (Hall, 1999) software is used for sequence comparing, the neighbor-joining method is used to construct phylogenetic relationship tree and drawn it with TreeView software.

After amplifying and sequencing the 16S rRNA gene sequence of LN strain (SEQ ID NO:3), choose the positions 54-510, including V1-V3 highly variable region, of nucleotide sequence 16S rDNA for comparing with other Bacillus genus bacteria species. The BioEdit Sequence Alignment Editor program (Hall, 1999) software is used for sequence comparing, and using the neighbor-joining method to construct phylogenetic relationship tree. The sequence comparing result showed in FIG. 2, the Bacillus amyloliquefaciens ATCC 23350 has the closest relationship with LN strain, their sequence similarity showed 99.9%.

According to the result of morphology observation, biochemical analysis and 16S rRNA gene provenance analysis above, LN strain was identified to belong to Bacillus amyloliquefaciens.

The Bacillus amyloliquefaciens LN strain isolated by present invention was tested for zearalenone removal ability and will describe below. The zearalenone concentration determined by the method described in Urraca et al. (2005), that is, using high performance liquid chromatography (HPLC) to determine. The C₁₈ reversed-phase chromatography column was used in HPLC analysis, the mobile phase was acetonitrile containing 15 mM ammonium acetate, methanol and water (10:55:35, v/v) mixture, the flow rate was 1.0 mL/min, and tested with fluorescence detector. The excitation wavelength and emission wavelength were set at 452 and 271 nm. The standard samples with varies zearalenone concentrations (0.5-8 ppm) were tested with fluorescence detector and integrated to making a calibration curve. The zearalenone concentrations of the samples were determined with the calibration curve.

To determine whether the zearalenone affect the growth of Bacillus amyloliquefaciens, 1% of Bacillus amyloliquefaciens LN and Bacillus amyloliquefaciens ATCC 23350 overnight culture seeded in LB broth with or without 3.5 ppm zearalenone, incubated at 37° C. with shaking at 250 rpm for 48 hours. Samples were taken at 0, 4, 8, 12, 24, 36 and 48 hours to determine the cell number using OD600 measurement.

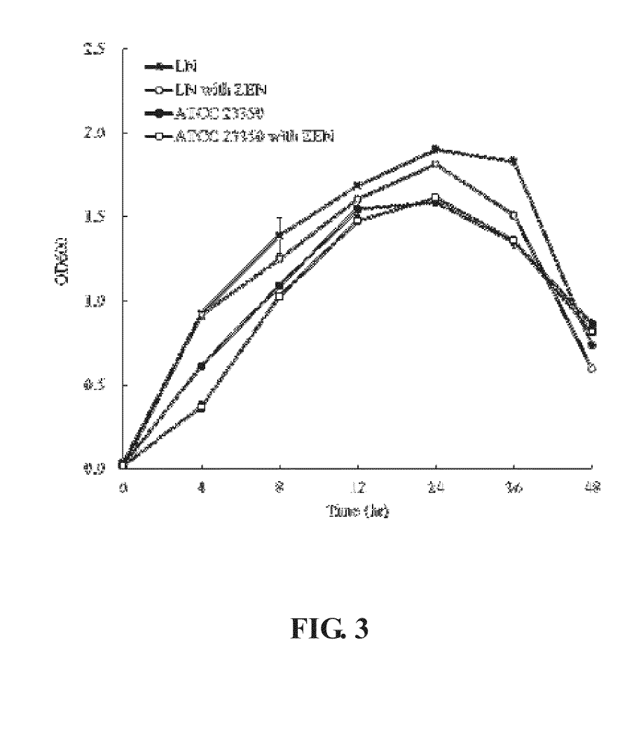

LN and ATCC 23350 strains seeded in LB broth at 37° C. with shaking, respectively. The two strains reached highest absorbance value at 1.90 and 1.68, respectively, indicating that LN strain had better growth status in LB broth. After incubating in LB broth containing 3.5 ppm zearalenone for 24 hours, the absorbance value of LN and ATCC 23350 strain is similar to which in LB broth without zearalenone, 1.81 and 1.62, respectly (FIG. 3). The result described above showed zearalenone doesn't affect the growth of LN and ATCC 23350 strain.

### Example 1

### Zearalenone Removal Ability of Bacillus amyloliquefaciens LN in LB Broth

To estimate the zearalenone removal ability of Bacillus amyloliquefaciens LN in LB broth, 1% of Bacillus amyloliquefaciens LN and Bacillus amyloliquefaciens ATCC 23350 seeded in LB broth with or without 3.5 ppm zearalenone, incubated at 37° C. with shaking at 250 rpm for 48 hours. Samples were taken at 0, 4, 8, 12, 24, 36 and 48 hours to purify and quantify the zearalenone concentration using the method described by Urraca et al. (2005).

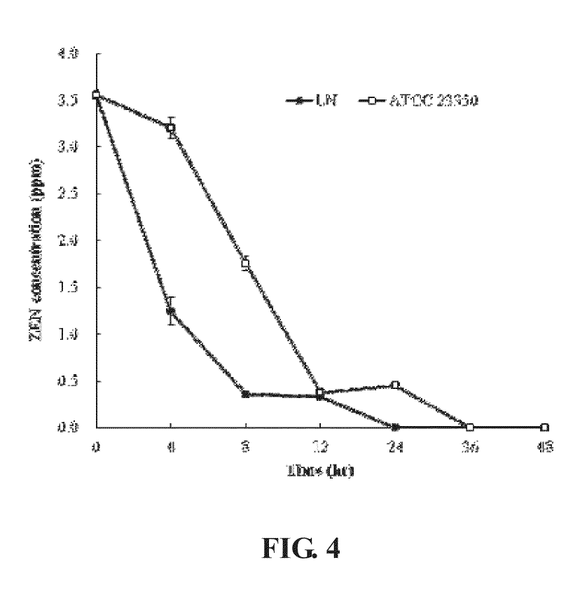

The result was shown in FIG. 4, LN strain removed all zearalenone in the LB broth after incubated for 24 hours, in contrast, ATCC 23350 strain removed all zearalenone in the LB broth after incubated for 36 hours. The result proved that LN strain has better zearalenone removal ability; the zearalenone decomposition rate is 100% in 24 hours.

### Example 2

### Zearalenone Removal Ability of Bacillus amyloliquefaciens LN in Phosphate Buffer Solution

1% of Bacillus amyloliquefaciens LN and Bacillus amyloliquefaciens ATCC 23350 overnight culture seeded in LB broth and incubated at 37° C. with shaking at 250 rpm for 24 hours, centrifuged with 8000 g for 20 mines to collect the cells. The cell resuspended in phosphate buffer solution containing 5 ppm zearalenone and adjusted the cell concentration to 10¹⁰ CFU/mL, incubated at 37° C. with shaking at 250 rpm for 48 hours. Samples were taken at 0, 4, 8, 12, 24, 36 and 48 hours to purify and quantify the zearalenone concentration using the method described by Urraca et al. (2005).

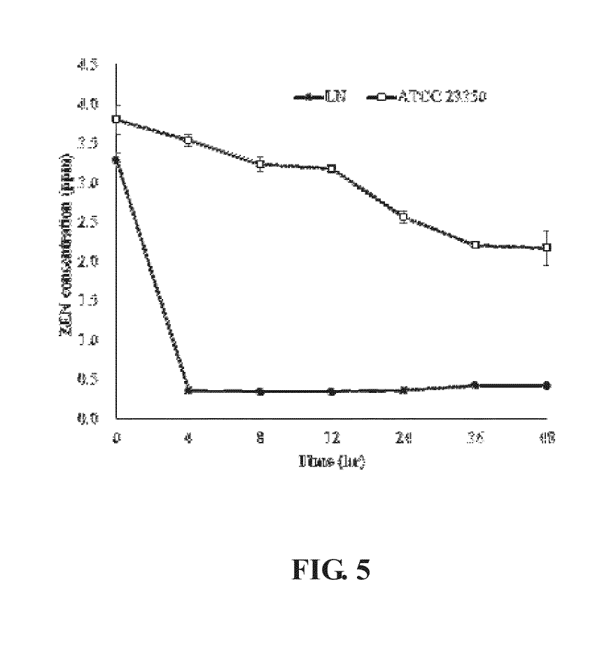

Further, to estimate the zearalenone removal ability of LN and ATCC 23350 strains in phosphate buffer solution. The 10¹⁰ CFU/mL cell suspended in phosphate buffer solution containing 5 ppm zearalenone and determined the zearalenone concentration in phosphate buffer solution during incubation. The result was shown in FIG. 5, the zearalenone concentration reduce to 3.28 ppm while LN strain added into the phosphate buffer solution containing 5 ppm zearalenone (0 hour), indicating the LN cell has zearalenone adsorption ability; after incubated for 4 hours, the zearalenone concentration reduce to 0.36 ppm, that is, 89% zearalenone was removed, indicating that LN strain has zearalenone decomposition ability. In ATCC 23350 strain's experiment, the zearalenone concentration only reduced to 3.80 ppm at 0 hour; after incubated 48 hours, the zearalenone concentration reduced to 2.17 ppm, that is, 42.19% zearalenone was removed, indicating that Bacillus amyloliquefaciens has zearalenone removal ability and LN strain is better than ATCC 23350 strain.

### Example 3

### Zearalenone Removing Effect of Bacillus amyloliquefaciens LN in Cornstarch

To estimate the zearalenone removing effect of Bacillus amyloliquefaciens LN in corn, 40 g cornstarch containing zearalenone suspended in 160 mL distilled water, sterilized at 121° C. and 1.5 atm for 15 minutes, the zearalenone concentration was determined at 1.56 ppm. Then, 1% LN strain overnight culture was seeded in, incubated at 37° C. for 48 hours and samples were taken at 0, 12, 24, 36 and 48 hours to measure zearalenone concentration. The cornstarch purchased from local supermarket and became zearalenone contaminated cornstarch by using the method of Paster et al. (1990).

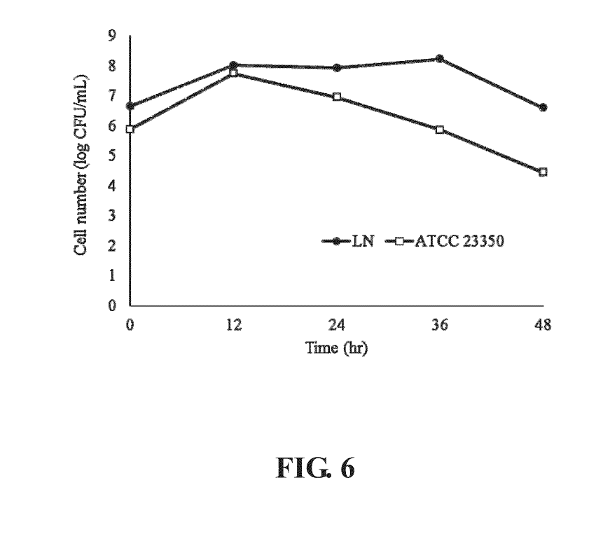

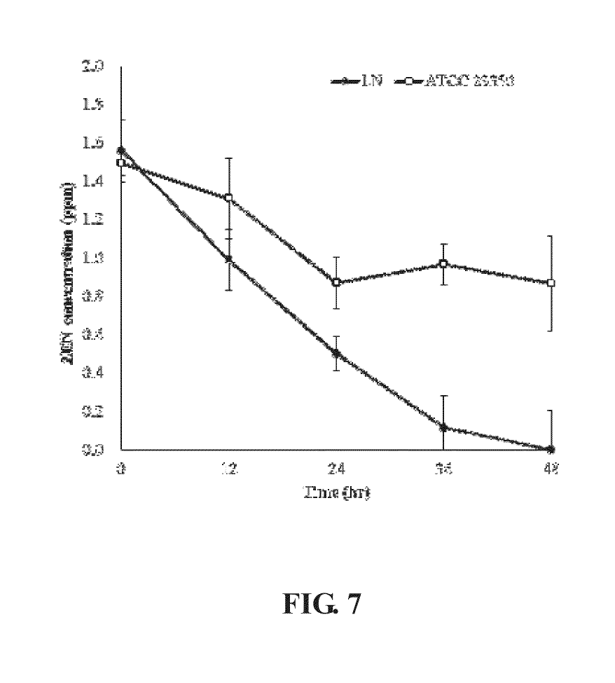

Using 20% w/w cornstarch containing 1.56 ppm zearalenone to incubate with LN strain, the cell number during incubation period was shown as FIG. 6. The initial cell number of LN strain was 6.66 log CFU/mL, then increased to 8.23 log CFU/mL after 36 hours incubation, indicating that LN strain could grow in cornstarch culture medium containing 1.56 ppm zearalenone. For the zearalenone concentration, that was 1.56 ppm before LN strain seeded in, and was 0.12 ppm after incubated 36 hours, indicating that the LN strain removed 92% zearalenone (FIG. 7).

### Example 4

### Safety Assessment of Bacillus amyloliquefaciens LN

This embodiment mainly estimated the hemolytic property, the antibiotics sensitivity and the enterotoxin producing property of LN strain, to provide the safety of the strain adding into a foodstuff or an ingredient and then be taken by human or animals.

### 4-1 Hemolytic Property of LN Strain

The LN strain was seeded in blood agar plate (Merck, Germany) and incubated at 37° C. for 24 hours to observe the colony morphology and to determine whether the hemolytic phenomenon is happened.

The colonies morphology of Bacillus amyloliquefaciens LN incubated on the blood agar plate were observed, there was no hemolytic zone around them, indicating that the LN strain has no hemolytic effect.

### 4-2 Sensitivity of Antibiotics of LN Strain

According to the method of Sorokulova et al. (2008), the LN strain overnight culture was diluted to 10⁶ CFU/mL and smeared on LB agar plate, then putting pastilles containing varies antibiotics (Mast Diagonstic, France). After incubated at 37° C. for 18 hours, we measured the diameter of inhibition zone and determined the sensitivity of antibiotics according to the method bulletined by Clinical and Laboratory Standards Institute (CLSI).

The result of antibiotics sensitivity assessment was shown as Table 2. LN strain showed no antibiotics sensitivity and resistance for ampicillin and streptomycin, but showed antibiotics sensitivity for other 18 antibiotics. In using bacteria having antibiotics resistance gene, the resistance gene may transfer to enteropathogens and resulting in make the enteropathogens drug resistance. Thus, the bacteria using for biochemical detoxication with no drug resistance is better (Salminen et al., 1998; Temmerman et al., 2002).

TABLE 2
Antibiotics sensitivity test of Bacillus amyloliquefaciens LN

| | | Zone diameter breakpoint | | |
|---|---|---|---|---|
| | Zone diam- | of CLSI standard (mm) | | |
| antibiotic | eter of LN | sensi- | inter- | resis- |
| (μg) | (mm)* | tivity | mediate | tance |
| Amikacin (30) | 23.0 ± 0.8 | ≧17 | 15-16 | ≦14 |
| Ampicillin (10) | 16.2 ± 1.0 | ≧17 | 14-16 | ≦13 |
| Chloramphenicol (30) | 30.7 ± 5.9 | ≧18 | 13-17 | ≦12 |
| Ciprofloxacin (30) | 25.0 ± 2.0 | ≧21 | 16-20 | ≦15 |
| Cefamandol (30) | 38.2 ± 7.1 | ≧18 | 15-17 | ≦14 |
| Cefoxime (30) | 28.7 ± 7.0 | ≧26 | 23-25 | ≦22 |
| Cefoxitin (5) | 27.6 ± 1.5 | ≧18 | 15-17 | ≦14 |
| Cephalothin (10) | 36.8 ± 1.7 | ≧18 | 15-17 | ≦14 |
| Erythromycin (15) | 24.5 ± 1.5 | ≧21 | 16-20 | ≦15 |
| Gentamicin (10) | 35.0 ± 9.6 | ≧15 | 13-14 | ≦12 |
| Imipeneem (10) | 29.5 ± 0.2 | ≧16 | 14-15 | ≦13 |
| Kanamycin (30) | 34.0 ± 0.4 | ≧18 | 14-17 | ≦13 |
| Neomycin (30) | 20.9 ± 2.3 | ≧18 | 14-17 | ≦13 |
| Nitrofurantoin (300) | 17.3 ± 0.6 | ≧17 | 15-16 | ≦14 |
| Norfloxacin (10) | 24.7 ± 0.8 | ≧17 | 13-16 | ≦12 |
| Oxacillin (1) | 14.3 ± 0.6 | ≧13 | 11-12 | ≦10 |
| Streptomycin (10) | 14.3 ± 0.5 | ≧18 | 15-16 | ≦14 |
| Tetracycline (30) | 15.3 ± 1.1 | ≧15 | 12-14 | ≦11 |
| Tobramycin (10) | 22.4 ± 1.8 | ≧15 | 13-14 | ≦12 |
| Vancomycin (30) | 19.0 ± 0.8 | ≧17 | 15-16 | ≦14 |

*Values are the means ± SD of triplicate measurements.

### 4-3 Enterotoxin Producing Property of LN Strain

Because of some of Bacillus genus bacteria, Bacillus cereus and Bacillus subtilis for instance, may produce enterotoxin which cause food poisoning (Lindback et al., 2004), and the gene of these enterotoxin may transfer to other bacteria (From et al., 2005). Thus, when using bacteria belong to Bacillus genus for biochemical detoxication, we should test the enterotoxin gene existence in the bacteria. There are two main enterotoxin which produced by Bacillus cereus: one is Hemolysin BL (Hbl), composed of subunit protein Hbl B, Hbl L₁, Hbl L₂ and etc. coded by gene hblA, hblB, hblC, hblD and etc., having hemolytic property, cytotoxicity and vascular permeability (Beecher et al., 2009); another one is nonhemolytic enterotoxin (Nhe), composed of subunit protein NheA, NheB, NheC and etc. coded by gene nheA, nheB, hblC, nheC and etc., mainly causing diarrhea (Lund and Granum, 1996). In addition, the enterotoxin T (BceT) coded by bceT gene having cytotoxicity, affecting vascular permeability and causing diarrhea (Agata et al., 1995). To determine whether the LN strain produces enterotoxin, enterotoxins were tested by PCR for detecting enterotoxin gene and by immune kit for detecting enterotoxin protein. The Bacillus amyloliquefaciens ATCC 23350, Bacillus cereus ATCC 11778 and Bacillus cereus ATCC 33019 purchased from Bioresource Collection and Research Center of Food Industry Research and Development Institute (Hsinchu, Taiwan) were used as the controls.

For detecting enterotoxin gene by PCR, according the method of Guinebretière et al. (2002) and Ouoba et al. (2007) to detect the existence of the hemolysin gene hblA, hblB, hblC, hblD, nonhemolytic enterotoxin gene nheA, nheB and nheC, enterotoxin gene bceT, etc. HAF (SEQ ID NO: 4) and HAR (SEQ ID NO: 5) were used for detecting hblA gene as the primer pair; HBF (SEQ ID NO: 6) and HBR (SEQ ID NO: 7) were used for detecting hblB gene as the primer pair; HCF (SEQ ID NO: 8) and HCR (SEQ ID NO: 9) were used for detecting hblC gene as the primer pair; HDF (SEQ ID NO: 10) and HDR (SEQ ID NO: 11) were used for detecting hblD gene as the primer pair; NAF (SEQ ID NO: 12) and NAR (SEQ ID NO: 13) were used for detecting nheA gene as the primer pair; NBF (SEQ ID NO: 14) and NBR (SEQ ID NO: 15) were used for detecting nheB gene as the primer pair; NCF (SEQ ID NO: 16) and NCR (SEQ ID NO: 17) were used for detecting nheC gene as the primer pair; BTF (SEQ ID NO: 18) and BTR (SEQ ID NO: 19) were used for detecting bceT gene as the primer pair.

For detecting enterotoxin gene by immunoassay kit, TECRA Bacillus Diarrheal Enterotoxin Visual Immunoassay kit (3M Co.) was used and followed the user's manual to detect whether LN strain produces Nhe enterotoxin protein.

To confirm the safety of the biochemical detoxification strain, the enterotoxin analysis is needed. We used PCR to detect whether the LN strain contains enterotoxin gene NheA, NheB, NheC, HblA, HblB, HblC, HblD, BceT, or etc. The results were shown in table 3, both of Bacillus amyloliquefaciens LN strain and Bacillus amyloliquefaciens ATCC 23350 contained no the gene fractions aforementioned, the control group of Bacillus cereus ATCC 11778 contains hblA, nheA, nheC, and bceT gene; Bacillus cereus ATCC 33019 contains hblA, hblB, hblC, hblD, nheA, nheB, nheC and bceT gene. Further, TECRA Bacillus Diarrheal Enterotoxin Visual Immunoassay kit was used to detect NheA subunit of Nheb enterotoxin. The result showed that both of Bacillus amyloliquefaciens LN strain and Bacillus amyloliquefaciens ATCC 23350 strain did not produce the enterotoxin protein.

TABLE 3
Results of enterotoxin detection

| | | | | | | | | BceT | Tecra |
|---|---|---|---|---|---|---|---|---|---|
| | Hbl enterotoxin ^{a} | | | | Nhe enterotoxin ^{a} | | | enterotoxin | kit test |
| strain | hblA | hblB | hblC | hblD | nheA | nheB | nheC | bceT1 | index ^{b} |
| B. amyloliquefaciens LN | − | − | − | − | − | − | − | − | 1 |
| B. amyloliquefaciens ATCC 23350 | − | − | − | − | − | − | − | − | 1 |
| B. cereus ATCC 11778 | − | − | − | − | + | + | + | + | 4 |
| B. cereus ATCC 33019 | + | + | + | + | + | + | + | + | 4 |

^{a} +: PCR positive; −: PCR negative.
^{b} according to the manual of the kit, score less than 3 represents negative.

### Example 5

### Probiotic Properties of Bacillus amyloliquefaciens LN Strain

When using bacteria to biochemical detoxify foodstuffs, the bacteria will enter the digestive system of the host with foodstuffs, thus the bacteria should be able to resist the acidic environment of stomach, survive in gastrointestinal, and detoxify the foodstuffs in gastrointestinal. Moreover, since the bacteria used for biochemical detoxification will enter the human or animals' body with foodstuffs, it's good for the host's gut health if the bacteria have probiotic properties. According to the guideline released by Food and Agriculture Organization of the United Nations (FAO), probiotics must have acidic resistant, bile salt resistant and anti-pathogenic properties.

### 5-1 Acidic Resistant Property

According to the method of Ehrmann et al. (2002), 1 mL overnight cultured LN strain or ATCC 23350 strain centrifuged at 8000 g, 4° C. for 20 minute, removed the supernatant and suspended the cells in 10 mL PBS (pH 2.0 or 3.0), took 0.1 mL cell suspension at 0, 0.5, 2 and 3 hours and serial diluted to appropriate dilution magnification, seeded in LB agar plates by spread plate technique and calculated the number of colonies after incubated at 37° C. for 24 hours.

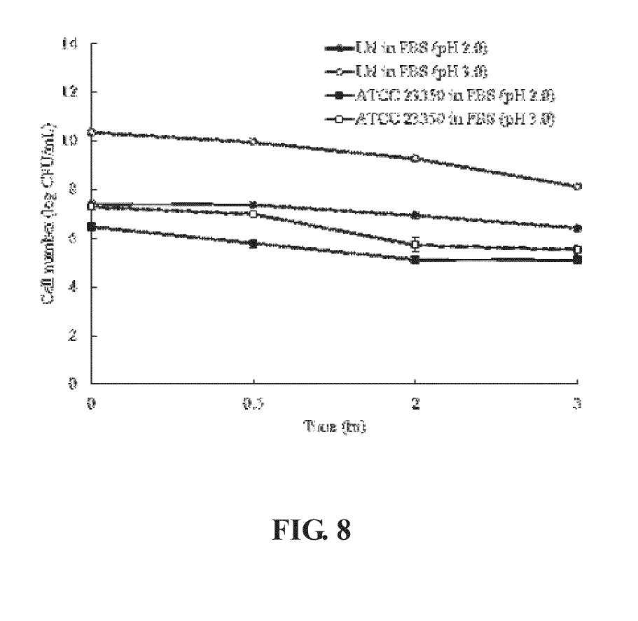

Using PBS with pH 2.0 or 3.0 to simulate the acidic environment in the stomach of animals, to detect whether the strain having acidic resistant property. The result showed in FIG. 8, the cell number of LN strain reduced to 6.4 log CFU/mL from 7.4 log CFU/mL within 2 hours while LN strain in pH 2.0 environment; the cell number of LN strain reduced to 8.1 log CFU/mL from 10.3 log CFU/mL within 2 hours while LN strain in pH 3.0 environment, proved that the LN strain have good acidic resistant property.

### 5-2 Bile Salt Resistant Property

According to the method of Ehrmann et al. (2002), 1 mL overnight cultured LN strain or ATCC 23350 strain seeded in 9 mL LB broth containing 0.3% oxgall at 37° C. for 24 hours, detected the absorbance at 600 nm at 0, 4, 8 and 12 hours during incubation.

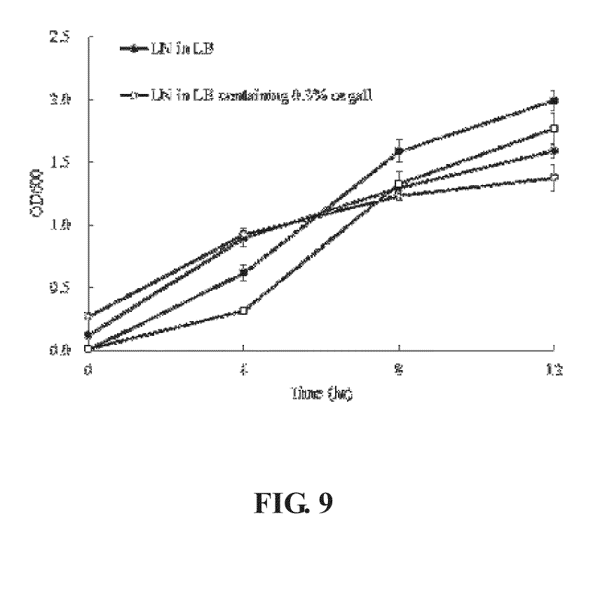

Using LB broth containing 0.3% oxgall to simulate the bile salt concentration in gut to detect whether the LN strain grows in such environment. The result showed in FIG. 9, after LN strain incubated in LB broth containing 0.3% oxgall for 12 hours, the absorbance was 1.38; the LN strain incubated in LB broth without bile salt for 12 hours, the absorbance was 1.5. This result proved that LN strain have bile salt resistant property, could keep growing in LB broth containing 0.3% bile salt.

### 5-3 Anti-Pathogenic Activities

The pathogens used for test including Salmonella enteri ATCC 12947, Listeria monocytogenes BCRC 14930, Listeria monocytogenes BCRC 15338, Listeria monocytogenes BCRC 15387, Bacillus cereus ATCC 11778, Bacillus cereus ATCC 33019 and Escherichia coli O157:H7, all pathogens were purchased from the Bioresource Collection and Research Center of Food Industry Research and Development Institute (Hsinchu, Taiwan).

According to the method of Yilmaz et al. (2006), overnight incubated pathogens coated on LB agar plates separately and dig a hole with 6 mm diameter. LN strain or ATCC 23350 strain overnight culture centrifuged at 8000 g for 20 minutes at 4° C., took the supernatant and filtered with 0.22 μm filter to remove bacteria, poured 40 μL filtered supernatant of LN strain or ATCC 23350 strain incubation medium into the hole on LB agar plates, measured the diameter of inhibition zones after incubated at 37° C. for 24 hours.

The anti-pathogenic activities test result for Bacillus amyloliquefaciens LN and ATCC 23350 strain showed in Table 4. Bacillus amyloliquefaciens LN strain inhibited the growth of Listeria monocytogenes BCRC 15338, B. cereus ATCC 11778 and ATCC 33019; in contrast, Bacillus amyloliquefaciens ATCC 23350 strain didn't show any inhibition effect in the tested pathogen.

TABLE 4
Anti-pathogen activities of Bacillus amyloliquefaciens
LN and ATCC 23350

| | Inhibition zone (mm) | |
|---|---|---|
| Species | LN | ATCC 23350 |
| B. cereus ATCC 11778 | 9.96 ± 0.14 | NI* |
| B. cereus BCRC 15850 | 8.64 ± 0.24 | NI |
| L. monocytogenes BCRC 14930 | NI | NI |
| L. monocytogenes BCRC 15338 | 10.63 ± 0.59 | NI |
| L. monocytogenes BCRC 15378 | NI | NI |
| S. enterica BCRC 12947 | NI | NI |
| E. coli O157:H7 | NI | NI |

*NI: no inhibition.
*Values are the mean ± standard deviations of triplicate measurements.

### Example 6

### Xylanase, Carboxyl-Methyl Cellulase, Amylase, and Protease Activities of Bacillus amyloliquefaciens LN Strain

If the bacteria used for biochemical detoxification has xylanase, carboxyl-methyl cellulase, amylase, and protease producing ability, then it could hydrolyze fiber and protein while detoxifying, thereby improve digestion and absorption of the foodstaffs or the feeding stuff.

1% LN strain or ATCC 23350 strain overnight culture seeded in 10 mL LB broth, incubated at 37° C. for 16 hours and centrifuged at 5000 g for 20 minutes at 4° C., collected the supernatant and determined the xylanase, carboxyl-methyl cellulase, amylase, and protease activities by enzyme diffusion method and enzyme specific activity assay.

### 6-1 Determine the Enzymes Activities by Enzyme Diffusion Method

According to the method of Waldeck et al. (2006), 15 mL agar gel containing 1% xylan, carboxymethyl cellulose or skimmed milk powder (1.5%) poured into a petri dish, and then dug out 6 mm diameter hole on the solidificated gel. LN strain or ATCC 23350 strain overnight culture centrifuged at 5000 g for 10 minutes at 4° C. and 40 μL supernatant was collected, putted into the hole on the agar gel and observed after overnight incubated at 37° C. for 16 hours. 10 mL congo red solution (0.75%) was added on the surface of agar gel containing xylan or carboxymethyl cellulose to stain for 15 minutes. The congo red solution after stained was removed, and then observed whether a light-color, clear ring existed around the hole, the existence of the clear ring indicating the added bacteria has xylanase or carboxyl-methyl cellulase activity. The agar gel containing skimmed milk powder could be observed without stain, and the existence of the clear ring around the hole indicating the added bacteria has protease activity.

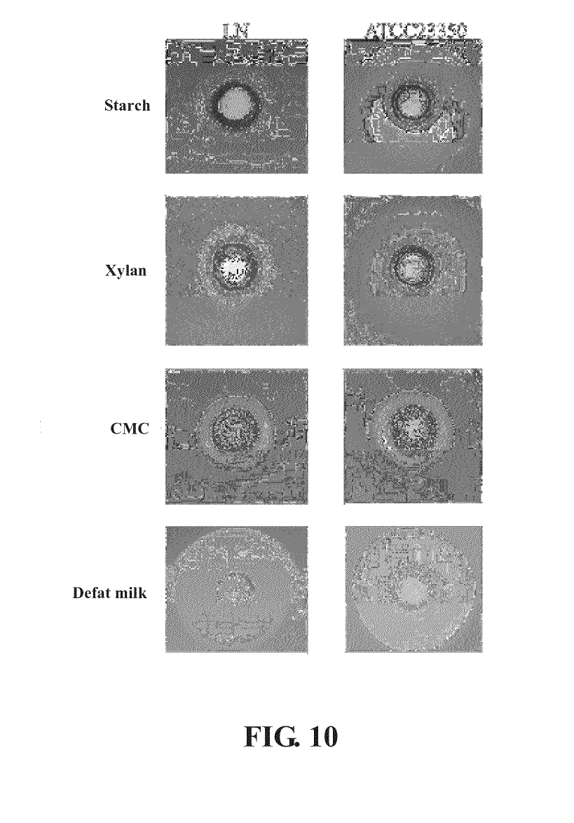

The result of the strain acted on the substrates showed in FIG. 10, the clear ring produced since the strain decomposed the substrate in the gel, indicating the strain has the enzyme activity for the substrate; Table 5 showed the enzyme diffusion result of Bacillus amyloliquefaciens LN strain and ATCC 23350 strain, the result demonstrated that both of the strains have the enzyme activity for carboxymethyl cellulose, skimmed milk powder, xylan and starch.

TABLE 5
Radial diffusion analyses of enzyme activity

Degradation zone (mm)

| Substrate | LN | ATCC 23350 |
|---|---|---|
| CMC | 12.67 ± 0.27* | 11.52 ± 0.75 |
| Xylan | 28.75 ± 1.51 | 27.83 ± 2.05 |
| Defat milk powder | 22.42 ± 1.00 | 22.82 ± 0.83 |
| Starch | 10.57 ± 0.57 | 20.80 ± 1.89 |

*All values are the means ± SD of triplicate measurements.

### 6-2 Enzyme Specific Activity Assay

The kit (Megazyme Co.; Wicklow, Ireland) for analyzed xylanase, carboxyl-methyl cellulase, amylase, and protease activities of the strain was used. The substrate is azo-xylan, azo-CMC or azo-casein. The instruction of manufacturer's manual was followed to detect the enzyme activities. LN strain or ATCC 23350 strain overnight culture centrifuged at 8000 g for 10 minutes at 4° C., diluted the supernatant for 10 times by sodium phosphate buffer solution (100 mM, pH 7.0) and then detected the enzyme activities. One unit of enzyme activity was defined as 1 μmol released dye per minute after acted with 1 mL enzyme.

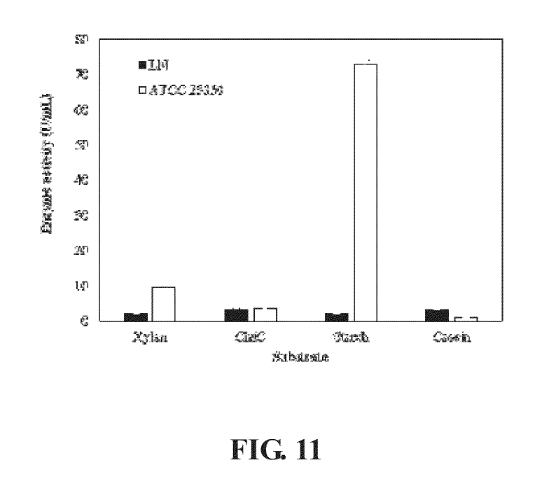

According to the method of Megazyme company to determine and quantify the enzyme activities for the substrates, xylan, carboxymethyl cellulose, starch and defat milk powder for instance. The result showed in FIG. 11, the enzyme activities of LN strain for xylan, carboxymethyl cellulose, starch and defat milk powder are 2.18±0.01 U/mL, 3.39±0.02 U/mL, 2.07±0.00 U/mL and 3.13±0.06 U/mL, respectively. The enzyme activities of ATCC 23350 strain for xylan, carboxymethyl cellulose, starch and defat milk powder are 9.71±0.01 U/mL, 3.60±0.01 U/mL, 72.84±0.02 U/mL and 1.02±0.06 U/mL, respectively. Thus, Bacillus amyloliquefaciens LN strain of the present invention is not only have zearalenone removal ability, but also have amylase, xylanase, carboxyl-methyl cellulase, and protease producing abilities.

According to the embodiments above, the present invention isolated a novel Bacillus amyloliquefaciens LN strain from a moldy corn sample. It has been confirmed that the LN strain belongs to the species Bacillus amyloliquefaciens in taxonomy through its morphological and biochemical characteristics and the result 16S rRNA gene sequencing. Bacillus amyloliquefaciens LN strain shows high zearalenone-degrading activity, and was demonstrated to be non-hemolytic, non-enterotoxin producing, and could survive in pH 2 or 3 acidic environment and bile salt containing medium. In addition, Bacillus amyloliquefaciens LN has xylanase, carboxyl-methyl cellulase, amylase, and protease activities. Therefore, Bacillus amyloliquefaciens LN is suitable for adding into the zearalenone contaminated feeding stuffs or food ingredients, thereby eliminating the hazard of zearalenone.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for detoxifying zearalenone by Bacillus amyloliquefaciens LN, comprising contacting a substance containing zearalenone with the Bacillus amyloliquefaciens LN, wherein the Bacillus amyloliquefaciens LN has been deposited under DSMZ Accession number DSM32119.

2. The method of claim 1, wherein the Bacillus amyloliquefaciens LN contacts with the substance containing zearalenone for a period of detoxification time.

3. The method of claim 2, wherein the period of detoxification time is 24-36 hours.

4. The method of claim 2, wherein the period of detoxification time is 24 hours and the zearalenone-degradation rate is 100%.

5. The method of claim 2, wherein the substance containing zearalenone is corn, wheat, rice, barley, millet or oats.

6. The method of claim 1, wherein the Bacillus amyloliquefaciens LN is supplemented into a food or an ingredient.

7. The method of claim 6, wherein the Bacillus amyloliquefaciens LN is supplemented into with 1% concentration.

8. The method of claim 1, wherein the Bacillus amyloliquefaciens LN is supplemented into a feeding stuff.

9. The method of claim 8, wherein the Bacillus amyloliquefaciens LN is supplemented into with 1% concentration.

10. A composition for removing zearalenone, comprising Bacillus amyloliquefaciens LN and an excipient, wherein the Bacillus amyloliquefaciens LN has been deposited under DSMZ Accession number DSM32119.
